(19) United States
(12) Patent Application Publication
(10) Pub. No.: US 2014/0342933 A1
(43) Pub. Date: Nov. 20, 2014

(54) METHODS AND COMPOSITIONS FOR MULTIPLEXED AND ULTRASENSITIVE MICRORNA DETECTION

(71) Applicant: Nesher Technologies, Inc., Los Angeles, CA (US)

(72) Inventors: Armin Reitmair, Irvine, CA (US); Taiho Kim, Los Angeles, CA (US); Steven Partono, Anaheim, CA (US)

(73) Assignee: Nesher Technologies, Inc., Los Angeles, CA (US)

(21) Appl. No.: 14/354,512

(22) PCT Filed: Oct. 26, 2012
(86) PCT No.: PCT/US12/62202

§ 371 Date: Apr. 25, 2014

Related U.S. Application Data
(60) Provisional application No. 61/552,404, filed on Oct. 27, 2011.

(51) Int. Cl. C12Q 1/68

(52) U.S. Cl. CPC C12Q 1/6886; C12Q 2600/158
USPC 506/9; 436/94

(57) ABSTRACT

Provided herein are methods and compositions for detection and quantification of one or multiple target miRNA(s) in biological fluids and/or tissue samples using alternating laser excitation (ALEX) single molecule fluorescence spectroscopy, and employing such methods and compositions for diagnostic, prognostic, therapeutic, and/or research applications.

### CROSS REFERENCE TO RELATED APPLICATION

This application claims the benefit of U.S. application Ser. No. 61/552,404, filed Oct. 27, 2011, which is hereby incorporated by reference in its entirety.

### GOVERNMENT INTERESTS

This invention was supported with U.S. government funds, NIH SBIR grant 1R43GM085962. The government therefore retains certain rights in the invention.

### BACKGROUND OF THE INVENTION

Prostate cancer is the second most common cancer among men in the United States. In 2007, 223,307 men were diagnosed with prostate cancer and 29,093 men died from prostate cancer. The risk of getting prostate cancer increases with age and it is estimated that 6.6% of men over the age of 60 will develop this cancer over the next 10 years.

The most common test for prostate cancer currently available is the PSA test, which measures the amount of prostate-specific antigen in the blood. The U.S. Food and Drug Administration (FDA) has approved the PSA test along with a digital rectal examination (DRE) for early detection of prostate cancer in men over the age of 50. PSA test reading of higher than 3.0 ng/ml is normally followed by a biopsy to confirm the presence of prostate cancer. However, PSA tests do not always provide accurate results. Studies have shown that between 65 and 75 percent of men with a positive PSA test result never develop prostate cancer during their lifetimes (lack of clinical specificity), while some men with a negative PSA test result were later diagnosed with prostate cancer (lack of clinical sensitivity).

In addition, PCA3 mRNA level can also be used for diagnosis of prostate cancer. In prostate cancer patients, an about 60-fold increase in PCA3 mRNA levels was observed when compared to normal patient samples. The PCA3 mRNA detection, however, involves collection of urine samples after DRE and thus presents discomfort to patients.

MiRNAs are small (18-25 nucleotides) non-coding RNAs that are important in regulating gene expression by binding to mRNA transcripts and influencing their stability or translation efficiency. These miRNAs have been shown to circulate within blood and appear to be relatively stable in the plasma and serum. Recently, miRNA expression profiles in certain cancers and diseases have been found to be altered, suggesting that some miRNAs, individually or as miRNA signatures, can be used as diagnostic and/or prognostic biomarkers, and/or as biomarkers to monitor responses to therapeutic interventions.

Recently, Mitchell et al. demonstrated significant overexpression of miR-141 in individuals with prostate cancer compared with normal individuals. At a miRNA-141 level of above 2,500 copies per μl of serum, individuals with prostate cancer were identified with 100% clinical specificity and 60% clinical sensitivity. They also demonstrated that miRNA levels in cancer individuals were moderately correlated with their PSA levels with Pearson and Spearman (rank) correlation coefficients of +0.85 and +0.62.

A list of miRNAs for diagnostic and prognostic uses for prostate diseases is provided in (US patent application, US 20100297652 A1 by Shelton, et al.).

Various nucleic acid assay technologies have been used to identify and characterize miRNAs, such as microarray- and polymerase chain reaction (PCR)-based assays. Particularly for miRNAs that are present in low amounts, amplification techniques such as quantitative real-time reverse transcriptase polymerase chain reaction (qRT-PCR) or isothermal NASBA, (nucleic acid sequence based amplification) has been used to amplify the targets of interest. However, immobilization of probes or target amplification decreases assay sensitivities and increases cost and time requirements.

Therefore, there is a critical need for a method that allows fast, sensitive, and inexpensive detection of multiple targets, including but not limited to microRNAs, mRNAs, ncRNAs, and DNAs, without amplification or pre-enrichment in a crude, unpurified sample where target concentrations are very low (less than 100 fM).

### SUMMARY OF THE INVENTION

The present invention relates to the technical field of medical diagnostics. One aspect provides a method for tumor marker detection. One aspect relates to the detection of specific microRNAs (miRNAs) such as miR-141, which has been known to be present in elevated concentrations in blood of prostate cancer patients, using fluorophore-labeled molecular-beacon probes and alternating laser excitation (ALEX) single molecule fluorescence spectroscopy. ALEX is described for example in U.S. Pat. No. 7,456,954 (Modulated Excitation Fluorescence Analysis), the contents of which are incorporated herein by reference.

The present invention describes methods and compositions for ultra-sensitive, highly multiplexed detection and quantification of low abundance biomolecules (less than 100 fM concentrations) including but not limited to microRNAs, mRNAs, ncRNAs, and DNAs in complex matrices such as bodily fluids and/or tissue samples using alternating laser excitation (ALEX) single molecule fluorescence spectroscopy.

Using the present method, amplified miRNA targets can be detected and quantified, either during or after the amplification process, using molecular beacon probes (U.S. Pat. No. 6,103,476, U.S. Pat. No. 5,925,517) labeled either with fluorescent dye-quencher pairs or fluorescent donor-acceptor FRET pairs (US 20090053821 A1, US 20050112673 A1, US 20030082547 A1, U.S. Pat. No. 7,803,536). Furthermore, due to the exquisite sensitivity of the single molecule detection approach, miRNA targets can be detected and quantified directly, without any amplification steps, with a limit of detection (LOD) of≧100 fM.

One aspect presented herein relates to a method of detecting and quantifying the amount of one or multiple target miRNA(s) in a biological fluid or tissue sample using ALEX single molecule fluorescence spectroscopy.

One embodiment relates to clinically ultra-sensitive and -specific tests for early detection, diagnosis, and/or prognosis of diseases in general, and cancer in particular, including but not limited to prostate cancer, by monitoring, individually or in a multiplexed fashion, a panel of relevant biomarkers, including but not limited to detecting elevated miR-141 levels in blood, other bodily fluids, and/or tissues. Using the method described herein it is possible to detect miR-141 spiked into serum at concentrations of 100 fM, while achieving a linear range of quantification over 2 log orders. This dynamic range is within clinically relevant concentrations of miRNAs in human serum. The panel of biomarkers may comprise any nucleic acid, including but not limited to miRNA, and/or any protein identifiable and quantifiable using ALEX single molecule fluorescence spectroscopy.

Examples for panels of miRNAs include miRNA signatures with prognostic (e.g. miR-486, -30d, -1, -499), and diagnostic (e.g. miR-25, -223, -335) potential for lung cancer.

Another aspect provided herein is direct detection and quantification of one or multiple target miRNA(s) without amplification. Currently, the most commonly used method for miRNA detection is qRT-PCR, which is a two-step process involving a cDNA synthesis step using a reverse transcriptase (RT), followed by amplification and quantification using real-time PCR. Due to their short sequences (18-25 nucleotides), the first step requires the use of looped RT primers which is challenging and can introduce amplification bias. The method described herein involves direct detection without the cDNA synthesis and amplification steps.

Another embodiment provides for performing amplification (e.g. isothermal NASBA) of miRNAs present at concentrations too low for direct detection, followed by molecular beacon probe(s) hybridization(s) and ALEX-based target(s) detection and quantification, either during or after amplification.

Another embodiment employs modified quenched locked nucleic acid (LNA)-based molecular beacon probes for ALEX-based miRNA detection and quantification. Locked nucleic acids are described for example in U.S. Pat. No. 7,060,809 B2 and U.S. Pat. No. 7,084,125 B2. Most miRNAs have strong secondary structures, and hence require hybridization at temperatures above their folding temperatures. Locked nucleic acids are nucleic acid analogues in which the ribose ring is “locked” by a methylene bridge connecting the 2′-O atom with the 4′-C atom, allowing a more rapid pairing with a complementary nucleotide strand and increasing the stability of the resulting duplex. The use of quenched molecular beacon probes, carrying one or multiple fluorophore(s) on one end and one or multiple quencher(s) at the other end, reduces background from non-hybridized, quenched probes and facilitates direct miRNA detection using ALEX single molecule fluorescence spectroscopy subsequent to target/probe hybridization and fluorophore signal dequenching.

Another embodiment provides a method implementing optimal hybridization conditions for miRNA detection and quantification, including but not limited to detection and quantification of miR-141, using modified quenched LNA-based molecular beacon probes as described above. Because both the miRNA and the complementary molecular beacon probe have secondary structures, the annealing process requires incubation at different temperatures such that 1) the target and the molecular probe maintain a linear conformation after the hybridization step for fluorophore signal dequenching and 2) the remaining free molecular beacon probes form a hairpin structure at a lower temperature for fluorophore quenching and thus background reduction.

Another embodiment provides for performing multiplexed detection and quantification of several miRNAs simultaneously. This extends one of the biggest advantages of using the ALEX detection scheme: the ability to detect multiple species in the same reaction mixture by incorporating multiple fluorescent dye probes with different excitation/emission characteristics in conjunction with multicolor excitation/detection to measure multiple distances between distinct fluorescence probes via FRET. Without FRET involvement between a donor fluorophore and an acceptor fluorophore, two color (2c) ALEX allows differentiation of two, three color (3c) ALEX differentiation of three, and four color (4c) ALEX differentiation of four miRNA species. Thus, in its simplest implementation for multiplexed miRNA detection and quantification, different emission wavelength signals are produced upon dequenching of individual dye-quencher pairs comprised of different wavelength fluorophore-quencher pairs.

To increase multiplexing power, distinct FRET values, specifically designed for each miRNA target, can be generated by placing multiple fluorophore-quencher pairs at distinct FRET distances on each probe. Moreover, by utilizing the full ES histogram (monitoring the probe stoichiometry ratio S and FRET efficiency E) through use of strong donor/weak acceptor (and/or weak donor/strong acceptor) dye pairs, the multiplexing capability can be increased further. It is also possible to detect miRNA targets in a multiplexed fashion without using probes with dye-quencher pairs, but with donor-acceptor FRET pairs that allow monitoring changes of E upon hybridization to target miRNA.

In general, multi-distance analysis towards more complex levels of n-color-ALEX will enable observation of n-component interactions up to [n(n-1)/2] donor-acceptor pairs and at least three (low, medium, and high FRET) multi-distances per FRET pair within a single biomolecule complex/target-sequence area, allowing full implementation of barcoding for highly multiplexed target detection in a single well.

Another embodiment provides a quick and simple method for miRNA detection in serum/plasma samples. The method employed here is using size exclusion columns for quick and simple extraction. Thus, the overall method can be divided into three simple steps: 1) initial purification of the serum/plasma sample using a size-exclusion filter, 2) hybridization and annealing with a molecular beacon probe, and 3) ALEX-based analysis. Using this method, we aim to detect and quantify miRNA targets in serum/plasma samples in less than 90 minutes, offering the prospect of using the method in point-of-care (POC) settings.

Another embodiment herein provides a combination of the method described herein with microfluidic chips, such as a modified version of the Formulator chip, for reduced sample and reagent requirements, automated sample handling as well as sequential miRNA detections in which a first batch of miRNAs is detected using a given number of molecular beacon probes, followed by detection of the next batch of miRNA using the same color but different sequence molecular beacon probes for the targets of interest in the second batch. Successful combination of microfluidics-based sample handling of nanoliter volumes with ALEX spectroscopy (“single molecule optofluidics”) has recently been demonstrated.

Another embodiment provides for an assay expansion to include more targets (e.g. single-well multiplexing power of≧10; e.g. total of≧100 miRNA targets) by implementing a multi-tiered analysis approach using e.g. 10 fluorophore/quencher-coded detectors with e.g. 10 mixed sequences each for the first round of analysis, followed by subsequent round(s) of analysis using e.g. 10 fluorophore/quencher-coded detectors with specific sequences corresponding to each code containing mixed sequences. The assay will be adjusted such that fluorescent signals will only be observed at or above clinically relevant threshold levels for each miRNA. This will allow monitoring a larger number of miRNAs simultaneously which should prove useful for assessing pharmacodynamic responses in context with drug efficacy evaluations during clinical trials.

Another embodiment provides a method for discovery of novel disease-related miRNA-based biomarkers using the principle of assay expansion as described above. Instead of using known miRNA sequences to synthesize molecular beacon-based probes, combinations of probes with novel sequences will be used to screen for elevated miRNA levels in body fluids obtained from diseased individuals versus healthy controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the predicted hairpin structure of miR-141 with a folding Tm of 72.2° C.

FIG. 2 illustrates the secondary structure of the molecular beacon probe in a quenched state. The + signs indicate where locked nucleic acids (LNAs) were incorporated.

FIG. 3 illustrates the predicted hybridization structure of miR-141 with the molecular beacon probe.

FIG. 4 illustrates the temperature steps and cycles used in the hybridization process of miR-141 with the molecular beacon probe.

FIG. 5 illustrates ALEX single molecule fluorescence spectroscopy used for measuring the fluorescent signals from hybridized probes (exemplified by four-color (4c) ALEX).

FIG. 6 shows ALEX-based measurement data generated from 1, 10, and 100 fM of synthetic miR-141 in buffer. A p-value of 0.006 (statistically highly significant) was calculated between buffer (B) and the 1 fM limit of detection (LOD) based on a 2-sided 2-sample Student's T-test for the null hypothesis of no difference (n=4).

FIG. 7 shows ALEX-based measurement data generated from 1, 10, and 100 fM of synthetic miR-141 spiked into 90% human serum. A p-value of 0.031 (statistically significant) was calculated between buffer (B) and the 1 fM limit of detection (LOD) based on a 2-sided 2-sample Student's T-test for the null hypothesis of no difference (n=4). Burst counts measured in unspiked 90% human control serum (0) reflect unknown endogenous miR-141 levels present in healthy individuals.

FIG. 8 illustrates a possible molecular beacon probe labeling scheme for multiplexed miRNA detection (exemplified for 3c-ALEX). Different signals are produced upon dequenching of individual dye-quencher pairs (A) or multiple fluorophore-quencher pairs positioned at distinct FRET distances between donor and acceptor (B).

FIG. 9 illustrates a possible molecular beacon probe labeling scheme for multiplexed miRNA detection without using dye-quencher pairs but with donor-acceptor FRET pairs that allow monitoring changes of FRET efficiency E upon hybridization to target miRNA.

FIG. 10 illustrates a microfluidic chip design for automated sample handling for sequential ALEX measurements.

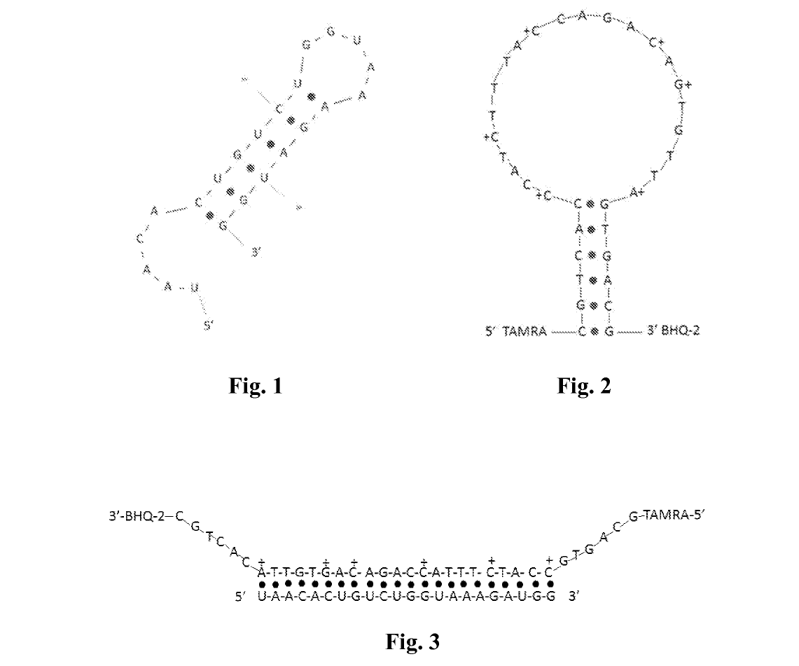

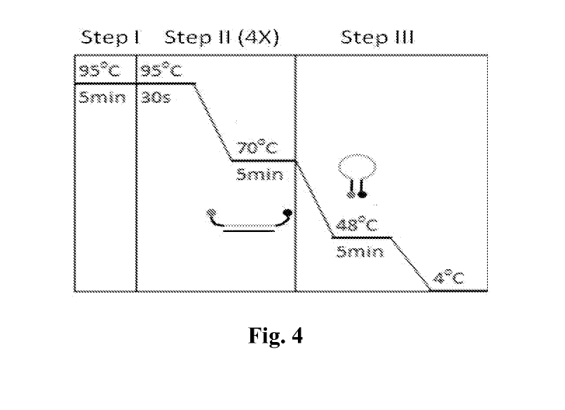

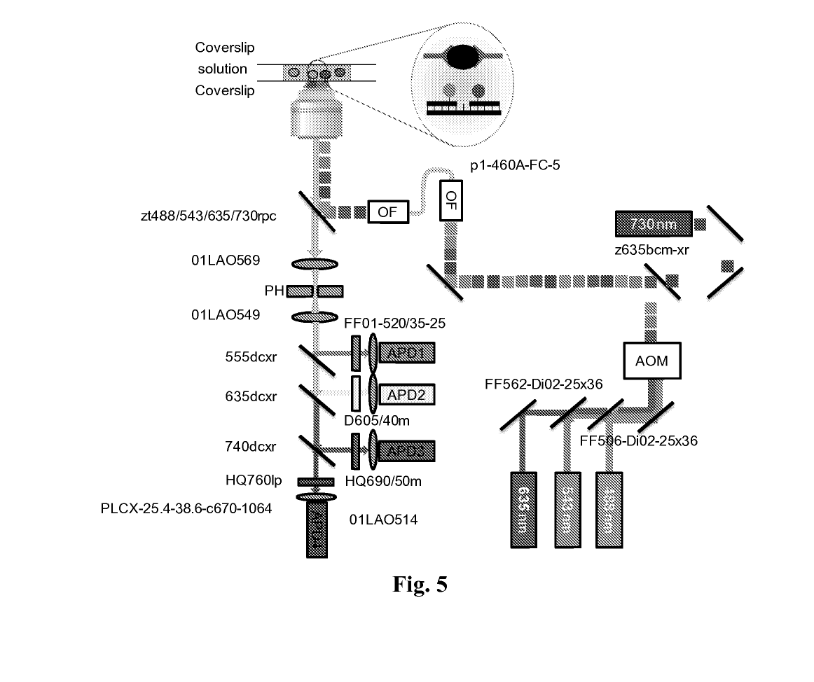

### DETAILED DESCRIPTION OF THE INVENTION

Provided herein, by way of example, are methods of detecting and quantifying miR-141 in a serum/plasma sample from an individual.

### Sample Preparation

Sample preparation involves purification of a serum/plasma sample using a size exclusion filtration step. E.g., 500 μl of serum/plasma is filtered through an Amicon centrifugal filter (10 kDa molecular weight cut-off) for 15 min at 14,000 g to separate the miRNA from most of large protein impurities. Sample sizes may range from 0.1 μl to 20 ml, and the size exclusion filtration step may be performed using microfluidics and/or centrifugation devices as described in the field. For miRNA analysis in cells or tissues, the miRNA needs to be isolated first using e.g. the mirVana™ PARIS™ Kit (life Technologies™).

### Hybridization Protocol

E.g., 50 μl of the pass through from the filtration step is hybridized with 1 nM of a LNA-based molecular beacon probe (5′-TAMRA/CGTCAC+CCAT+CTTTA+CCAGA+CA+GTGTT+AGTGACG/3′-BHQ2) in 10 mM Tris pH 8.0, 1 mM EDTA, 250 mM NaCl and subjected to the following temperature cycle: Step I involves incubation at 95° C. for 5 minutes to denature the miRNA and the molecular beacon probe. Step II involves slow cooling from 95° C. to 70° C. at a ramp rate of 0.1° C. per second, followed by incubation at 70° C. for 5 minutes. To facilitate annealing of the probe with the target, this step is repeated four times. Step III involves slow cooling from 70° C. to 48° C. followed by incubation at 48° C. for 5 minutes to facilitate refolding and quenching of the excess molecular beacon probes.

Detection of miR-141 in Hybridization Buffer

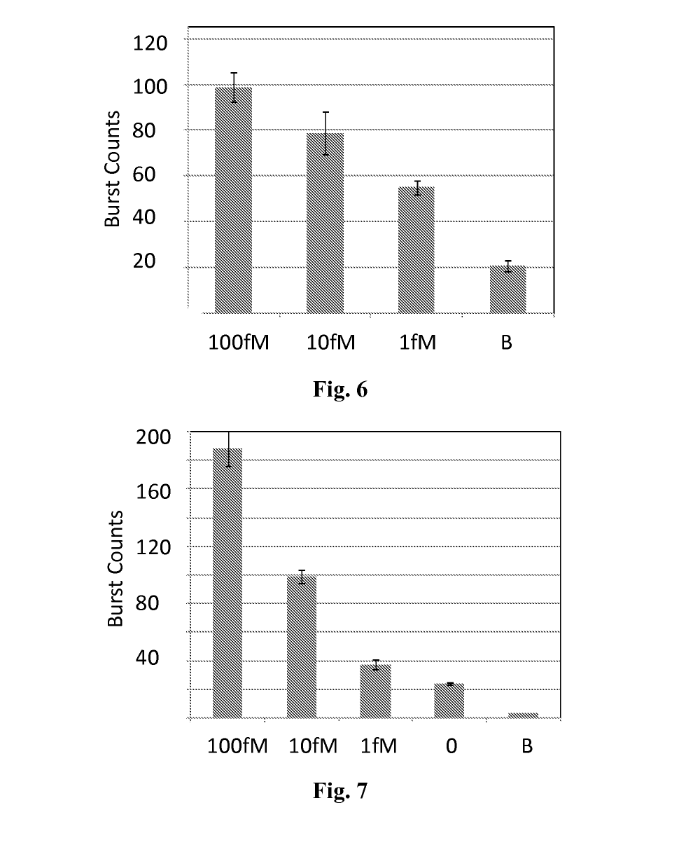

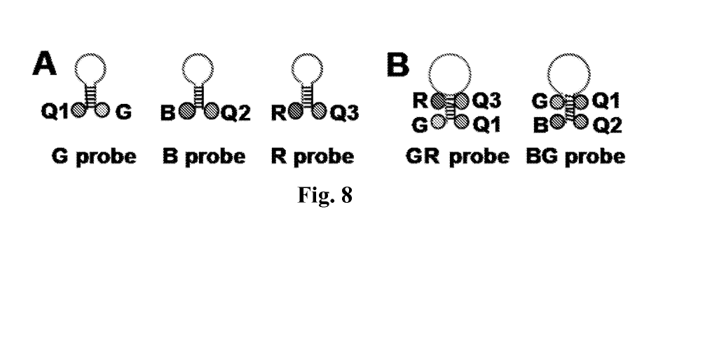

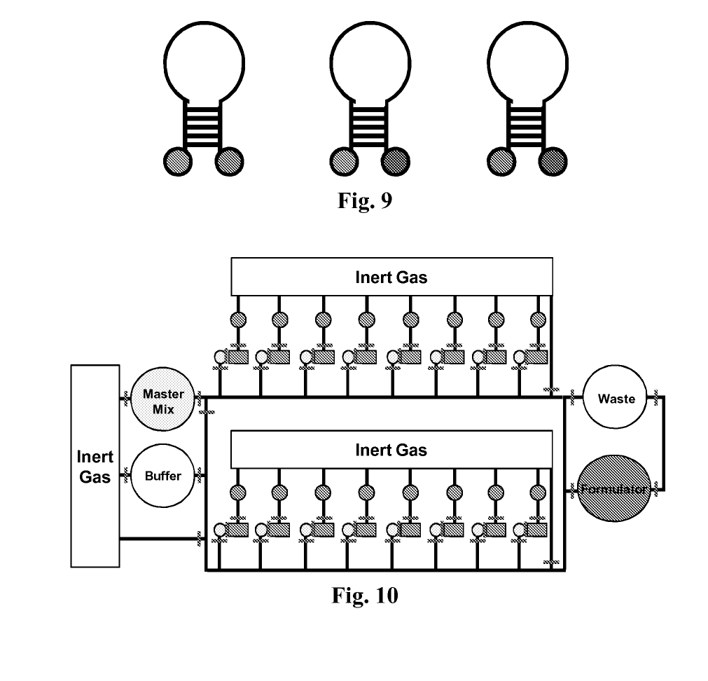

To demonstrate detection of clinically relevant concentrations of miR-141, we hybridized a synthetic miR-141 (5′-UAACACUGUCUGGUAAAGAUGG-3′) at various concentrations (1 fM, 10 fM, and 100 fM) with 1 nM labeled probe using the hybridization protocol described above. The hybridized probe was detected with 3c-ALEX single molecule fluorescence spectroscopy using a continuous 532 nm laser at 75 μW for 10 min The burst counts observed are shown in FIG. 6.

Detection of miR-141 spiked into 90% human serum 50 μl of target miRNA were added into 450 μl of human serum, and the mixture was filtered through an Amicon centrifugal filter (10 kDa molecular weight cut-off) for 15 min at 14,000 g to separate most of large protein impurities. 50 μl of the pass-through were diluted into 100 μl hybridization buffer containing 1 nM of the probe, followed by hybridization and detection as described above. The burst counts observed are shown in FIG. 7.

### Probes, Dyes, Quenchers

Examples of probes that can be used are LNA (U.S. Pat. No. 7,060,809) and stem-loop molecular beacons (U.S. Pat. Nos. 6,103,476 and 5,925,517). Examples of dyes that can be used are HiLyte Fluor™ Dyes (ANASPEC), Alexa dyes (Invitrogen), Cy dyes (GE Healthcare), Atto dyes (ATTO-TEC), and Dylight dyes (Thermo Scientific). Examples of quenchers that can be used are QXLTM quenchers (ANASPEC) and black hole quenchers (BHQ, BIOSEARCH Technologies).

### Example of a Multiplexed Microrna and Protein Biomarker-Based Next-Generation Prostate Cancer Test

The use of PSA for early detection and staging of prostate cancer as well as monitoring responses to surgical, hormonal and radiation therapy is well established. However, one of the major limitations is false-positive results and there is an urgent need to increase the diagnostic specificity of prostate cancer tests, while maintaining—or even increasing—sensitivity. As for a growing number of different cancer types panels of multiple tumor markers are now being identified as more informative than individual markers, combination of a miRNA signature with traditional protein markers could translate into a better diagnostic tool with much higher clinical specificity, sensitivity, and accuracy, suitable for population-wide screening efforts.

MiRNAs may have great diagnostic potential for cancer and the potential to revolutionize present clinical management, including determining cancer classification, estimating prognosis, predicting therapeutic efficacy, maintaining surveillance following surgery, as well as forecasting disease recurrence. Of particular interest are tumor-specific circulating stable miRNAs as noninvasive biomarkers for different tumor entities. As described above, Mitchell et al. evaluated the expression of miR-141 in a case-control cohort of serum samples. At serum levels of >2510 copies per microliter, individuals with cancer were detected with 100% clinical specificity (however, only at 60% clinical sensitivity). NTI achieved ALEX-based detection of 600 miR-141 copies/μl (1 fM; see FIGS. 6 and 7), offering the prospect of direct detection and quantification of miRNAs at clinically relevant concentrations without any amplification. (In comparison, a recently published single-molecule method by US Genomics for the quantification of miRNA showed assay sensitivity of only 500 fM). In addition to miR-141, two more miRNA's with significantly elevated levels in prostate cancer patient sera, miR-125b and miR-375, are to be included in the miRNA marker panel to improve robustness of the test.

To further improve robustness of the test, several protein-based markers are to be included in the test. Serum PSA exists in different molecular forms. Its main portion is complexed mostly with alpha-1-antichymotrypsin (ACT), as well as with alpha-protease inhibitor (API), or alpha-2-macroglobulin (AMG). A smaller portion (5-35%) is free PSA. The ratio of free-to-total PSA has become an important variable in addition to total serum PSA levels for discriminating between men with prostate cancer and without apparent prostatic disease, or with benign prostatic hyperplasia (BPH). As there is an ever increasing list of other potential prostate cancer protein markers to improve specificity (as many as 91 were cited by Tricoli et al.), the following promising markers shall be included in the proposed panel (in addition to measuring free and complexed PSA): Chromogranin A (CgA), a marker of neuro-endocrine differentiation with elevated levels being associated with disease progression, poor prognosis and hormone-refractory disease, and human glandular kallikrein 2 (hK2), which is elevated in prostate cancer and also in BPH.

The completely solution-based, amplification-free assay will allow fast, accurate, and ultrasensitive quantification of cancer-related miRNA signatures and protein markers in small sample sizes (e.g. finger-prick), and overcome limitations of current detection technologies. (E.g. real-time PCR may introduce quantification bias due to target amplification and has limited multiplexing potential due to excitation/emission wavelength limitations; Microarray-based expression analysis requires large amounts of RNA and sensitivities are limited).

By combining markers with high clinical specificity (e.g. miRNA-141) and sensitivity (e.g. PSA), the proposed test, which ultimately will require only a drop of blood obtainable from a finger-prick, is expected to significantly reduce over-diagnosis and over-treatment of prostate cancer, while concomitantly offering considerable cost-savings, with the potential of eliminating the digital rectal exam (DRE) which is currently performed in conjunction with PSA testing.

The appealing aspect of the single-molecule approach, in particular when combined with microfluidics-based sample handling, is the ability to use very low amounts of precious reagents and patient samples. The exquisite sensitivity associated with single-molecule detection to record subtle changes of target concentrations in ultra-small volumes allows considerable cost and patient sample savings. As ALEX permits detection of both nucleic acid- and protein-based targets with the same instrumentation, further cost savings are expected for end-users due to reduction of required lab equipment to perform different types of tests.

What is claimed is:

1. A method for detecting and quantifying the amount of one or multiple target miRNA(s) in a biological fluid and/or tissue sample; the method comprising detecting miRNA by alternating laser excitation (ALEX) single molecule fluorescence spectroscopy; with or without prior miRNA isolation, amplification, or washing steps, with a limit of detection (LOD) of≦100 femtomolar (fM).

2. The method of claim 1 wherein the method provides clinically ultra-sensitive and -specific tests for early detection, diagnosis, and/or prognosis of a disease.

3. The method of claim 2 wherein the disease is a cancer.

4. The method of claim 3 wherein the cancer is prostate cancer, and the method comprises monitoring, in a multiplexed fashion, a panel of relevant biomarkers.

5. The method of claim 4 wherein the method comprises detecting elevated miR-141 levels in serum, plasma, blood, other bodily fluids, and/or tissues.

6. The method of claim 4 wherein the panel of biomarkers comprises a nucleic acid, including but not limited to miRNA, and/or any protein, and/or any nucleic acid identifiable and quantifiable using ALEX single molecule fluorescence spectroscopy.

7. The method of claims 1 comprising development of a multiplexed miRNA and protein biomarker-based prostate cancer test monitoring a panel of biomarkers present in blood.

8. The method of claim 7 wherein the panel comprises one or more of microRNAs: miR-141, miR-125b, and miR-375; proteins: PSA (free as well as complexed with alpha-1-antichymotrypsin (ACT), alpha-protease inhibitor (API), or alpha-2-macroglobulin (AMG)), Chromogranin A (CgA), and human glandular kallikrein 2 (hK2).

9. The method of claim 1 comprising direct detection and quantification of one or multiple target miRNA(s) without amplification.

10. The method of claim 1 comprising detection and quantification of one or multiple target miRNA(s) subsequent to or concomitant with target amplification.

11. A method comprising use of modified quenched locked nucleic acid (LNA)-based molecular beacon probes (and/or modified LNA-based FRET probes) for ALEX-based miRNA detection and quantification.

12. A method comprising implementing optimal hybridization conditions for ALEX-based miRNA detection and quantification.

13. The method of claim 12 comprising detection and quantification of miR-141, using modified quenched LNA-based molecular beacon probes (and/or modified LNA-based FRET probes).

14. The method of claim 13 comprising incubation at different hybridization temperatures such that 1) the target(s) and the molecular probe(s) maintain a linear conformation after the hybridization step for fluorophore signal dequenching (and/or for low FRET signals) and 2) the remaining free molecular beacon probes form a hairpin structure at a lower temperature for fluorophore quenching (or the remaining free FRET probes form either a hairpin structure or a random coil structure for high FRET signals).

15. The method of claim 1 comprising performing multiplexed detection and quantification of several miRNAs simultaneously by incorporating multiple fluorescent dye probes with different excitation/emission characteristics in conjunction with multicolor excitation/detection to measure multiple distances between distinct fluorescence probes via FRET, allowing full implementation of barcoding for highly multiplexed target detection in a single well.

16. The method of claim 1 comprising miRNA detection in blood samples comprising the steps: 1) initial preparation of serum/plasma samples, 2) purification of the serum/plasma sample using a size-exclusion filter, 3) hybridization and annealing with a molecular beacon probe, and 4) ALEX-based analysis.

17. The method of claim 1 further comprising use of microfluidic chips for reduced sample and reagent requirements, automated sample handling as well as sequential miRNA detections.

18. The method of claim 1 comprising development of expanded assays to include more targets (e.g. single-well multiplexing power of≧10; e.g. total of≧100 miRNA targets) by implementing a multi-tiered analysis approach using e.g. 10 fluorophore/quencher-coded detectors with e.g. 10 mixed sequences each for the first round of analysis, followed by subsequent round(s) of analysis using e.g. 10 fluorophore/quencher-coded detectors with specific sequences corresponding to each code containing mixed sequences. The assay will be adjusted such that fluorescent signals will only be observed at or above clinically relevant threshold levels for each miRNA.

19. The method of claim 1 comprising discovery of novel disease-related miRNA-based biomarkers using the principle of assay expansion.

20. The method of claim 19 comprising using combinations of probes with novel sequences to screen for elevated miRNA levels in body fluids obtained from diseased individuals versus healthy controls.


SEQUENCE LISTING (3 sequences)

SEQ ID NO: 1 - DNA, 34 bp, Artificial Sequence
  Description of Artificial Sequence Synthetic probe
cgtcacccatctttaccagacagtgttagtgacg

SEQ ID NO: 2 - RNA, 22 nt, Artificial Sequence
  Description of Artificial Sequence Synthetic oligonucleotide
uaacacugucugguaaagaugg

SEQ ID NO: 3 - DNA, 34 bp, Artificial Sequence
  Description of Artificial Sequence Synthetic probe
gcagtgccatctttaccagacagtgttacactgc